(19) United States
(12) Patent Application Publication
(10) Pub. No.: US 2024/0304944 A1
(43) Pub. Date: Sep. 12, 2024

(54) IONIC LIQUID ELECTROLYTES BASED ON FLUORINATED ALCOHOLIC BORATES AND ALUMINATES

(71) Applicant: Sumitomo Chemical Co., Ltd, Tokyo (JP)

(72) Inventors: Thomas Johannes Kugler, Godmanchester (GB); Antonio Attanzio, Godmanchester (GB)

(21) Appl. No.: 18/562,736

(22) PCT Filed: May 19, 2022
(86) PCT No.: PCT/EP2022/063636

§ 371 Date: Nov. 20, 2023

(30) Foreign Application Priority Data
May 19, 2021 (GB) 2107162.6
Mar. 31, 2022 (GB) 2204673.4

(51) Int. Cl. H01M 50/426; C07C 29/70; C07F 5/06; H01M 10/0525

(52) U.S. Cl. CPC H01M 50/426; C07C 29/70; C07F 5/069; H01M 10/0525

(57) ABSTRACT

A compound of formula (I): (I) wherein X is Al or B: R¹ in each occurrence is independently a substituent; and two R¹ groups may be linked to form a ring; and M⁺ is a cation. The compound may be used in a metal ion battery or metal battery.

### BACKGROUND

CN101771166 discloses an ionic liquid electrolyte composed of certain organic lithium borate or lithium aluminate compounds and certain organic compound containing an amido functional group.

JP2004265785 discloses an ionic electrolyte material of formula (I):

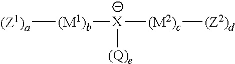

JP 2006/107793 discloses an ion having a fluorinated alkoxy group coordinated to a metallic element.

JP03409852 discloses compounds of formula:

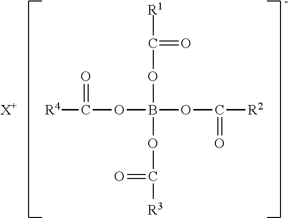

U.S. Pat. No. 8,394,539 discloses lithium salts with fluorinated chelated orthoborate anions used as electrolytes or electrolyte additives in lithium-ion batteries. The lithium salts have two chelate rings formed by the coordination of two bidentate ligands to a single boron atom.

E. Zygadło-Monikowska et al, “Lithium conducting ionic liquids based on lithium borate salts”, Journal of Power Sources 195 (2010) 6055-6061, discloses reaction of trialkoxyborates with butyllithium to form Li{[CH₃(OCH₂CH₂)nO]₃BC₄H₉}.

Michael Rohde et al, “Li[B(OCH₂CF₃)₄]: Synthesis, Characterization and Electrochemical Application as a Conducting Salt for LiSB Batteries”, ChemPhysChem 2015, 16, 666-675 discloses formation of Li[B(OCH₂CF₃)₄] by reaction of lithium borohydride with excess 2,2,2-trifluorethanol

R Tao et al, “Enhancement of ionic conductivity by mixing lithium borate with lithium aluminate”, discloses compounds of formula:

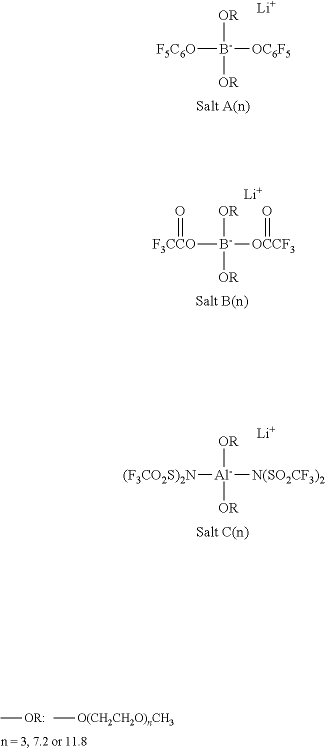

Andreas Thum et al, “Solvate ionic liquids based on lithium bis(trifluoromethanesulfonyl)imide-glyme systems: coordination in MD simulations with scaled charges” Phys. Chem. Chem. Phys., 2020, 22, 525-535 discloses equimolar mixtures of lithium bis(trifluoromethanesulfonyl)imide (Li[NTf₂]) with triglyme or tetraglyme.

Daniel J. Eyckens and Luke C. Henderson, “A Review of Solvate Ionic Liquids: Physical Parameters and Synthetic Applications”, Frontiers in Chemistry, April 2019, Vol 7, Article 263 is a review of solvate ionic liquids.

Seki, S., Takei, K., Miyashiro, H., and Watanabe, M “Physicochemical and electrochemical properties of glyme-LiN(SO2F)2 complex for safe lithium ion secondary battery electrolyte”, J. Electrochem. Soc. 158, A769-A774 (2011) discloses electrolyte performance of a CH₃—(OC₂H₄)₃—CH₃ (TG)/LIN(SO₂F)₂ (LIFSI) 1:1 molar mixture for lithium-ion secondary batteries.

### SUMMARY

In some embodiments, the present disclosure provides a compound of formula (I):

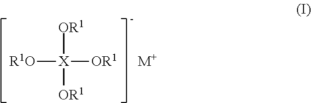

wherein X is Al or B; R¹ in each occurrence is independently a substituent; and two R¹ groups may be linked to form a ring; and M⁺ is a cation.

In some embodiments, none of the R¹ groups are linked. According to these embodiments, optionally each R¹ is independently a C₁₋₂₀ alkyl group wherein one or more non-adjacent C atoms of the alkyl group may be replaced with O, S, CO or COO and one or more H atoms of the alkyl group may be replaced with F.

Optionally each R¹ is independently selected from alkyl and alkyl ether groups wherein one or more H atoms may be replaced with F.

In some embodiments, each R¹ is the same.

In some embodiments, the compound of formula (I) contains at least 2 different R¹ groups.

In some embodiments, R¹ groups of formula (I) are linked and the compound of formula (I) has formula (Ia):

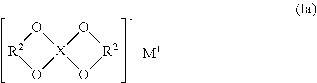

wherein R² in each occurrence is independently a divalent organic group.

Optionally, M⁺ of the compound of formula (I) is an alkali metal ion, optionally a lithium ion.

Optionally, M⁺ is a solvated cation.

Optionally the solvent of the solvate is selected from solvents comprising at least one ether group.

In some embodiments, the present disclosure provides a formulation comprising or consisting of a solvent and a compound of formula (I) wherein the formulation contains no more than 10 moles of solvent, more preferably no more than 7 moles or no more than 6 moles of solvent, per mole of M⁺.

Some or all of the solvent present in a formulation may be solvating solvent. The amount of solvating solvent in a compound of formula (I) may be determined from a ¹H NMR spectrum of the compound following vacuum treatment to remove free (non-solvating) solvent by integration of ¹H NMR peaks corresponding to the solvent and peaks corresponding to the groups —O—R¹.

In some embodiments, the present disclosure provides a method of forming a compound of formula (I) comprising reacting a compound of formula (II) and at least one compound selected from formulae (IIIa) and (IIIb):

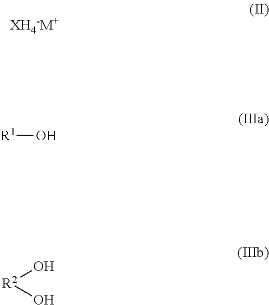

Optionally, the compounds of formula (IIIa) are selected from monohydric alcohols and mono-carboxylic acids and the compounds of formula (IIIb) are selected from diols and di-carboxylic acids.

Optionally, M⁺ of the compound of formula (I) is solvated and the reaction is carried out in a reaction mixture comprising the solvent of the solvate.

In some embodiment, the present disclosure provides a method in which M⁺ of the compound of formula (I) is solvated, the method comprising at least partially replacing the solvent of the solvate with another solvent.

In some embodiments, the present disclosure provides a polymer comprising a repeat unit of formula (IV):

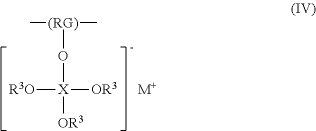

wherein RG is a repeating group of the polymer; R³ is a substituent; and X and M⁺ are as described above.

In some embodiments, the present disclosure provides a metal battery or metal ion battery comprising an anode, a cathode and a compound of formula (I) or polymer as described herein disposed between the anode and the cathode.

Optionally, the battery is a metal battery comprising an anode protection layer comprising the compound or polymer disposed between the anode and cathode.

### DESCRIPTION OF DRAWINGS

FIG. 1 is a schematic illustration of a battery according to some embodiments of the present disclosure having a separator comprising a compound as described herein;

FIG. 2 is a schematic illustration of a battery according to some embodiments of the present disclosure having an anode protection layer comprising a compound as described herein;

FIG. 3 is a NMR spectrum of a compound according to an embodiment of the present disclosure formed by reaction in THF;

FIG. 4 is an NMR spectrum of a compound according to an embodiment of the present disclosure formed by reaction in dimethoxyethane (DME);

FIGS. 5-9 are NMR spectra of the compound of FIG. 4 following addition of varying amounts of DME;

FIG. 10 is the cyclic voltammetry plot for the compound of FIG. 3 in which the compound was disposed between a copper foil working electrode and a lithium foil counter electrode;

FIG. 11 is an image of lithium deposited on the copper foil described in FIG. 10;

FIG. 12 is a Nyquist plot of the compound of FIG. 3.

FIG. 13 is Nyquist plots of the compounds of FIGS. 5-9; and

FIG. 14 is a plot of ionic conductivity vs. DME:Li cation ratio.

The drawings are not drawn to scale and have various viewpoints and perspectives. The drawings are some implementations and examples. While the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to an element of the Periodic Table include any isotopes of that element.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

In some embodiments, the present disclosure provides compounds of formula (I):

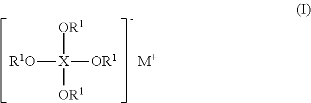

X is Al or B.
R¹ in each occurrence is independently a substituent and two R¹ groups may be linked to form a ring.
M⁺ is a cation.

In some preferred embodiments, none of the R¹ groups are linked. Optionally according to these embodiments, each R¹ is independently a C₁₋₂₀ alkyl group wherein one or more non-adjacent C atoms of the alkyl group may be replaced with O, S, CO or COO and one or more H atoms of the alkyl group may be replaced with F.

Preferred R¹ groups include C₁₋₂₀ alkyl wherein one or more C atoms other than the C atom bound to O of OR¹ or a terminal C atom may be replaced with O, and one or more H atoms may be replaced by F.

By “terminal C atom” of an alkyl group as used herein is meant the C atom of the methyl group or methyl groups at the chain end or chain ends of a linear or branched alkyl, respectively.

In some embodiments, each R¹ is the same.

In some embodiments, the compound contains two or more different R¹ groups.

In some embodiments, R¹ groups of formula (I) are linked and the compound of formula (I) has formula (Ia):

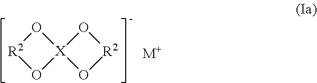

wherein R² in each occurrence is independently a divalent organic group.

Optionally, R² is selected from a C₆₋₂₀ arylene group, e.g. 1,2-phenylene, which may be unsubstituted or substituted with one or more substituents; a bi-arylene group, for example 2,2′-linked biphenylene; ethylene; and propylene, each of which may be unsubstituted or substituted with one or more substituents. Optionally, substituents are selected from F alkyl wherein one or more non-terminal C atoms of the C₁₋₁₂ alkyl may be replaced with F and one or more C atoms of the C₁₋₁₂ alkyl may be replaced with O.

The compound of formula (I) may be a liquid at 25° C. and 1 atm. pressure.

Preferably, M⁺ is an alkali metal cation, more preferably a lithium cation.

Preferably, M⁺ is a solvated cation.

Preferably, the solvent of the solvate is selected from solvents comprising at least one ether group.

Preferably, the solvent contains two or more groups capable of coordinating to the metal cation.

The solvent may be selected from linear and cyclic compounds containing one or more ether groups and, optionally, one or more groups selected from hydroxyl and carboxylate groups.

Exemplary solvents include, without limitation, tetrahydrofuran, dimethoxyethane, diglyme (diethylene glycol dimethyl ether), triglyme (triethylene glycol dimethyl ether), tetraglyme (tetraethylene glycol dimethyl ether) and crown ethers, for example 12-Crown-4 and 1-aza-12-Crown-4.

The compound may contain more than one solvent of a solvate.

Optionally, a battery containing a compound of formula (I) contains no solvent, or only a small amount of solvent, preferably no more than 10 moles of solvent per mole of M⁺. The presence of a small amount of solvent has been found to significantly increase the ionic conductivity of the compound of formula (I). This increase is attributed to solvation of the cation; where solvation takes place, it will be understood that M⁺ is solvated by at least some but not necessarily all of the solvent present. The presence of a small amount of organic solvent such as an ether-containing solvent may enhance ionic conductivity whilst significantly reducing flammability as compared to an ionic compound dissolved in a large volume of such a solvent.

Accordingly, a formulation containing or consisting of a solvent and a compound of formula (I) preferably comprises no more than 10 moles of solvent, more preferably no more than 7 moles or no more than 5 moles of solvent, per mole of M⁺. Preferably, the compound contains at least 0.5 moles or at least 1 mol of solvent per mole of M⁺. The formulation may contain only one solvent compound or may contain a mixture of two or more solvent compounds.

The compound of formula (I) may be formed by reacting a compound of formula (II) and at least one compound selected from formulae (IIIa) and (IIIb):

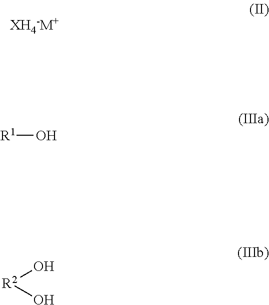

It will be understood that the compounds of formulae (IIIa) and (IIIb) may be selected according to the desired R¹ and R² groups of formula (I).

Exemplary compounds of formula (I) include, without limitation, lithium aluminium hydride (LiAlH₄), lithium borohydride (LiBH₄)

Exemplary compounds of formula (IIIa) include, without limitation:

C₁₋₂₀ alkyl monohydric alcohols which may be non-fluorinated or wherein one or more, optionally all, H atoms of the C₁₋₂₀ alkyl may be replaced with F, for example ethanol, isopropanol, 1H,1H,5H-octafluoro-1-pentanol and pentadecafluoro-1-octanol;
ether monohydric alcohols which may be non-fluorinated, partially fluorinated or perfluorinated for example 2-ethoxyethanol, diethylene glycol monoethyl ether, 1H,1H-perfluoro-3,6-dioxaheptan-1-ol, 1H,1H-perfluoro-3,6,9-trioxadecan-1-ol, 1H,1H-perfluoro-3,6-dioxadecan-1-ol and 1H,1H-perfluoro-3,6,9-trioxatridecan-1-ol; and
mono-carboxylic acid compounds, for example C₁₋₂₀ alkyl carboxylic acids wherein one or more non-adjacent C atoms other than terminal C atoms or the C atom adjacent to the carboxylic acid group may be replaced with O and one or more H atoms may be replaced with F. Examples include, without limitation: perfluoroalkyl carboxylic acids, for example trifluoroacetic acid; perfluoroalkyl ether carboxylic acids; and alkyl ether carboxylic acids.

Exemplary compounds of formula (IIIb) include alkane diols wherein one or more non-adjacent, non terminal C atoms other than the C atom bound to O of R²—O may be replaced with O; aromatic diols; dicarboxylic acids; and compounds having one hydroxyl and one carboxylic acid group, each of which may be unsubstituted or substituted with one or more substituents, optionally non-fluorinated, partially fluorinated or perfluorinated.

Exemplary compounds of formula (IIIb) include ethylene glycol, catechol (1,2-dihydroxybenzene), oxalic acid and fluorinated derivatives thereof.

In some embodiments, the reaction is carried out with only one compound selected from compounds of formulae (IIIa) and (IIIb). According to these embodiments, the R¹ groups (and, therefore, each R² group in the case of compounds of formula (II)) are all the same.

In some embodiments, the reaction is carried out with two or more compounds selected from compounds of formulae (IIIa) and (IIIb). According to these embodiments, the R1 groups may be different. The ratio of different R¹ groups may be selected according the ratio of the compounds of formulae (IIIa) and (IIIb) and their relative reactivity.

If the metal cation M⁺ is a solvated cation then in some embodiments the solvent of the solvate is present in the reaction mixture containing the compound of formula (II) and the compound of formula (IIIa) and/or (IIIb).

In some embodiments, the solvent of a compound of formula (I) containing a solvated cation may be replaced with a different solvent. Methods of changing the solvent of a solvate include, without limitation, driving off a solvent of a compound of formula (I) by heat treatment and replacing it with another solvent capable of solvating the cation; and contacting a compound of formula (I) with a solvent which coordinates more strongly to the cation than an existing solvating solvent, for example by treating a compound of formula (I) having a monodentate solvate solvent with a bi-dentate or higher-dentate solvate solvent.

In some embodiments, the present disclosure provides a polymer comprising a repeat unit of formula (IV):

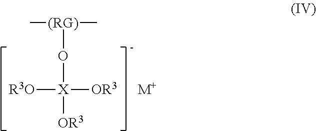

wherein RG is a repeating group of the polymer; R³ is a substituent; and X and M⁺ are as described above.
R³ may be a polymeric chain or a substituent R¹ as described above.

The polymer may be formed by reacting a compound of formula (II) as described above with a starting polymer having a backbone repeating group substituted with a hydroxyl or carboxylic acid group. The reaction may be performed in the presence of a compound of formula (IIIa) or (IIIb); the ratio of polymer:non-polymer groups may be selected according to the ratio of the starting polymer to the compounds of formula (IIIa) and/or (IIIb) and their relative reactivities.

The polymer may be formed by reacting a compound of formula (I) as described above with a starting polymer.

The starting polymer may be, for example, cellulose, optionally in a power or fibrous form.

### Applications

A single-ion conducting compound of formula (I) as described herein may be provided in a rechargeable battery cell. The battery may be, without limitation, a metal battery or a metal ion battery, for example a lithium battery or a lithium ion battery.

The compound of formula (I) may be a component of a composite comprising one or more additional materials, for example one or more polymers. A composition comprising a compound of formula (I) and a polymer may form a gel.

A layer comprising or consisting of the compound of formula (I) may be formed by depositing a formulation containing the material dissolved or dispersed in a solvent or solvent mixture. Optionally, a battery comprising the compound of formula (I) contains no more than 10 moles of solvent per mole of M⁺ and/or no solvent other than any solvating solvent.

The formulation may comprise a polymer additional material dissolved in the solvent or solvents.

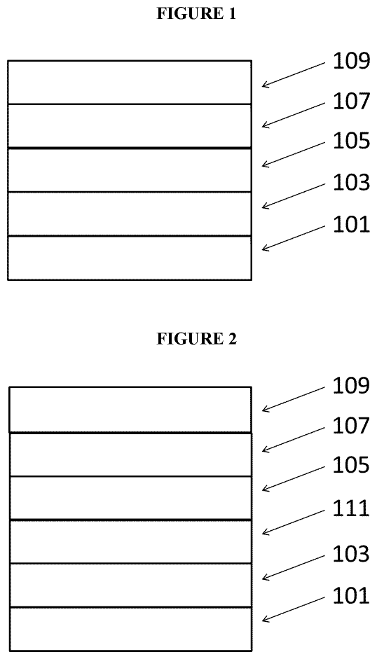

FIG. 1 illustrates a battery comprising an anode current collector 101 carrying an anode 103 on a surface thereof; a cathode current collector 109 having a cathode 107 disposed on a surface thereof; and a separator 105 disposed between the anode and cathode. The separator comprises or consists of a compound of formula (I). Preferably, the separator comprises no more than 10 moles of solvent per mole of M⁺ and/or no solvent other than any solvating solvent as described herein.

The battery may be a metal battery. The battery may be a metal ion battery.

In the case of a metal battery, the anode is a layer of metal (e.g. lithium) which is formed over the anode current collector during charging of the battery and which is stripped during discharge of the battery.

In the case of a metal ion battery, the anode comprises an active material, e.g. graphite, for absorption of the metal ions.

The cathode may be selected from any cathode known to the skilled person.

The anode and cathode current collectors may be any suitable conductive material known to the skilled person, e.g. one or more layers of metal or metal alloy such as aluminium or copper.

FIG. 1 illustrates a battery in which the anode and cathode are separated only by a separator. In other embodiments, one or more further layers may be disposed between the anode and the separator and/or the cathode and the separator.

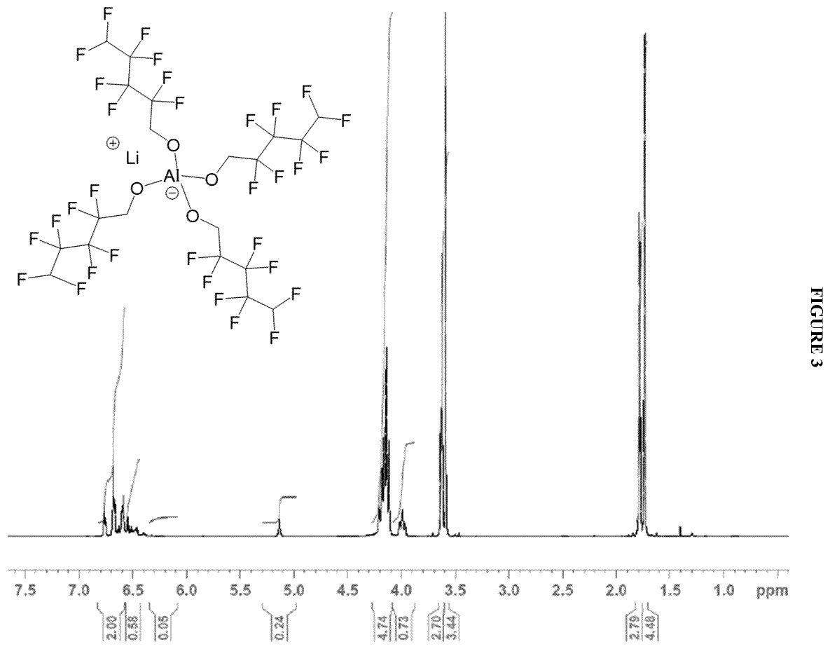

FIG. 2 illustrates a battery, preferably a metal battery, comprising an anode current collector 101 carrying an anode 103 on a surface thereof; a cathode current collector 109 having a cathode 107 disposed on a surface thereof; a separator 105 disposed between the anode and cathode; and an anode protection layer 111 disposed between anode and the separator. The separator may comprise or consist of a compound as described herein or may be any other separator known to the skilled person, for example a porous polymer having a liquid electrolyte absorbed therein. The anode protection layer comprises or consist of a compound of formula (I) as described herein. The anode protection layer may prevent or retard formation of lithium metal dendrites of a metal battery.

### EXAMPLES

### Compound Example 1—Synthesis 1

Compound Example 1 was prepared according to Scheme 1.

2.15 ml of a solution of LiAlH₄ in THF (1 M) was added dropwise to a stirred solution of 1.19 ml of 2,2,3,3,4,4,5,5-octafluoropentan-1-ol (OFP) in dry THF (2 ml). The solution bubbled during the addition. The rate of addition was controlled to prevent excess bubbling and heating. The mixture was then stirred at room temperature for 2 hours.

The excess solvent was then removed under reduced pressure (3.0×10⁻² mbar for 30 mins). During this time the mixture was stirred to disperse the bubbles formed by the evaporating solvent. The mixture slowly concentrated into a thick oil, and then into a viscous liquid, stopping the stirrer bar. The sample was left under vacuum for 5 more minutes, before the evacuated flask was sealed and transferred to the glovebox.

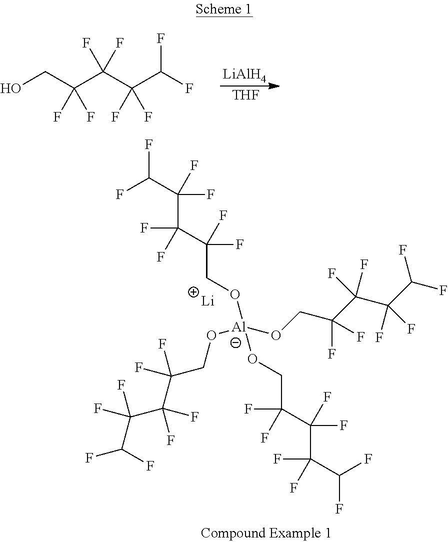

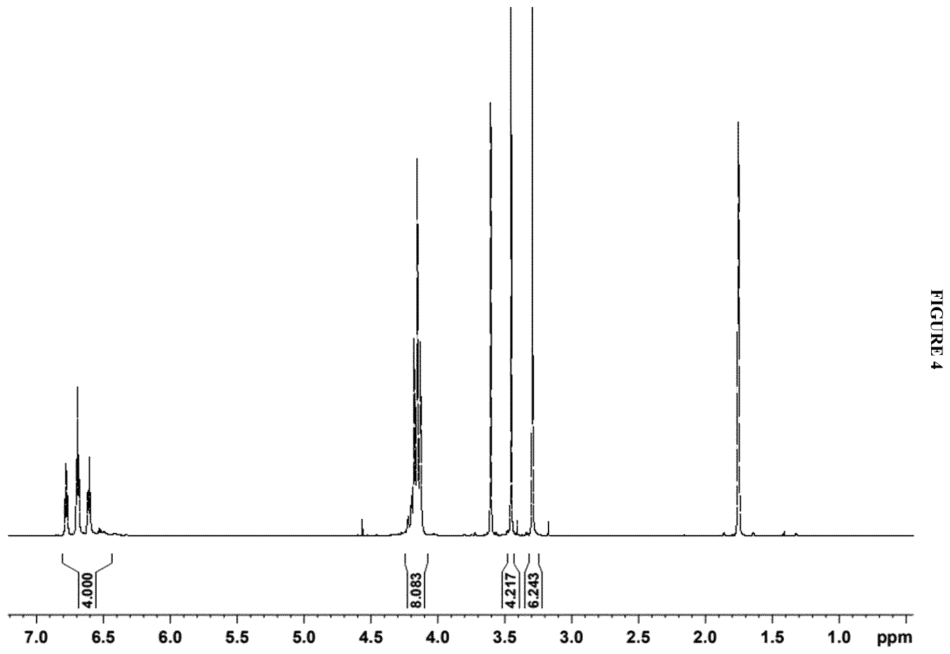

The NMR spectrum of the product in deuterated THF is shown in FIG. 3. The spectrum shows that there are potentially two product species containing OFP units (6.2-6.8 and 4.2 ppm) and that about 10% of unreacted OFP is still present in the mixture (5.1 and 4 ppm).

From integration of NMR peaks, it was calculated that for four molecules of OFP in the products mixture there is one molecule of THE present as residual solvent, at least some of which is believed to form part of a solvate.

### Compound Example 1—Synthesis 2

The reaction was performed as described in Synthesis 1

To a solution of OFP (5 ml, 35.8 mmol) in anhydrous 1,2-dimethoxyethane (1,2-DME) (10 ml), a solution of lithium aluminium hydride (9 ml, 9.0 mmol, 1.0M in tetrahydrofuran) was added between 5° C. and room temperature. The resulting mixture was stirred at room temperature for 30 minutes, then heated to 60° C. for 30 minutes. The excess solvent was removed under reduced pressure (3.0×10−2 mbar) at 25° C. for 4 hours to yield a thick gel.

¹H NMR (600 MHZ) in deuterated THF: δ (ppm), 3.29 (s, CH₃ from 1,2-DME, 6.24H), 3.45 (s, CH₂ from 1,2-DME, 4.22H), 4.16 (t, CF₂CH₂, J=14 Hz, 8H), 6.69 (tt, CF₂CF₂H, J=51.6 Hz, J=5.9 Hz, 4H).

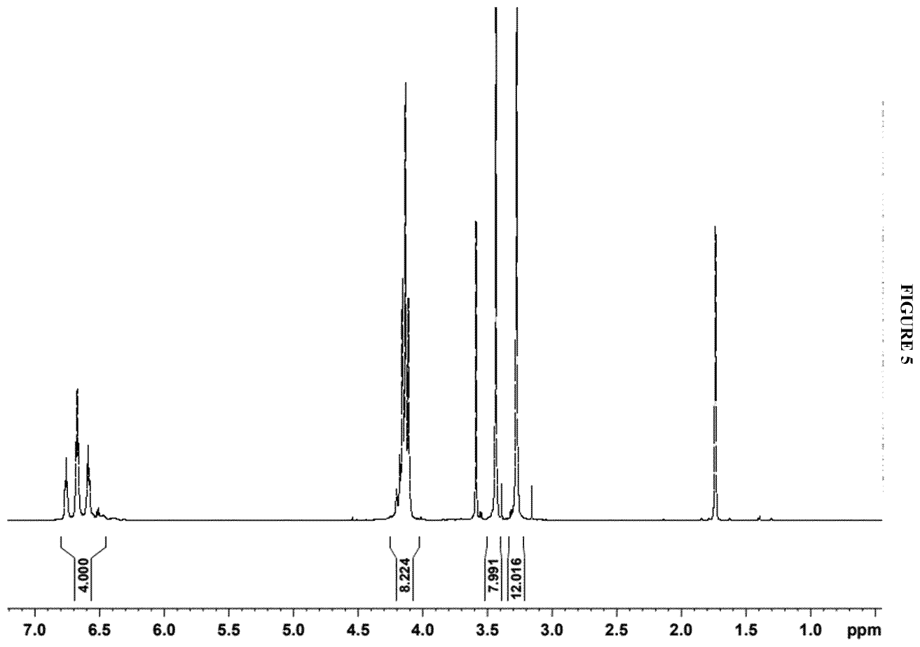

From integration of NMR peaks (FIG. 4), it was calculated that for four molecules of OFP in the product, which correspond to one lithium cation there is 1.05 molecules of 1,2-DME present as residual solvent.

### Preparation of Electrolyte

In a glovebox filled with Argon gas, LiAl(OFP)₄ obtained from Synthesis 2 as a soft gel was weighed into 20 ml bottles. Different amounts of 1,2-dimethoxyethane were then added to the gels (see table 1) and bottles were capped. Bottles were sonicated and placed on roller for up to 1 hour to obtain homogenous transparent liquids. The electrolyte entry 5 in table 1 was obtained by dissolving the remaining material stuck on the walls of the flask containing the parent LiAl(OFP)4 with 0.3 ml of 1,2-DME.

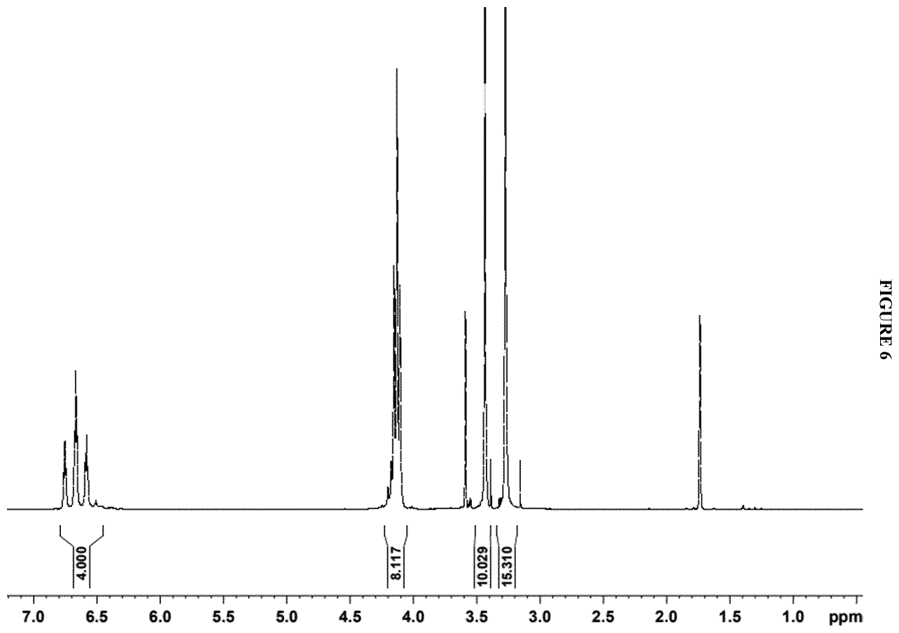

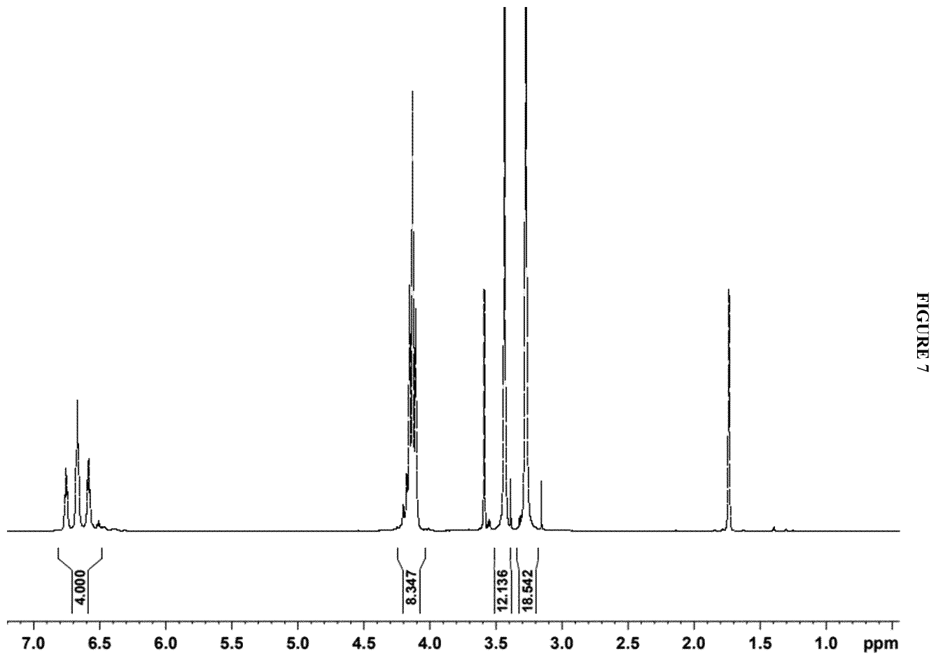

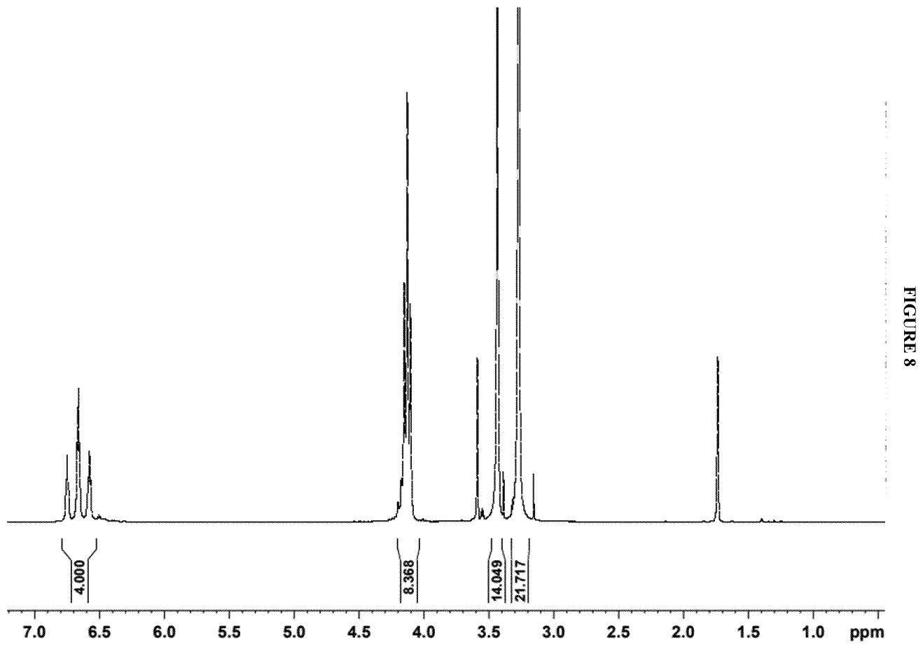

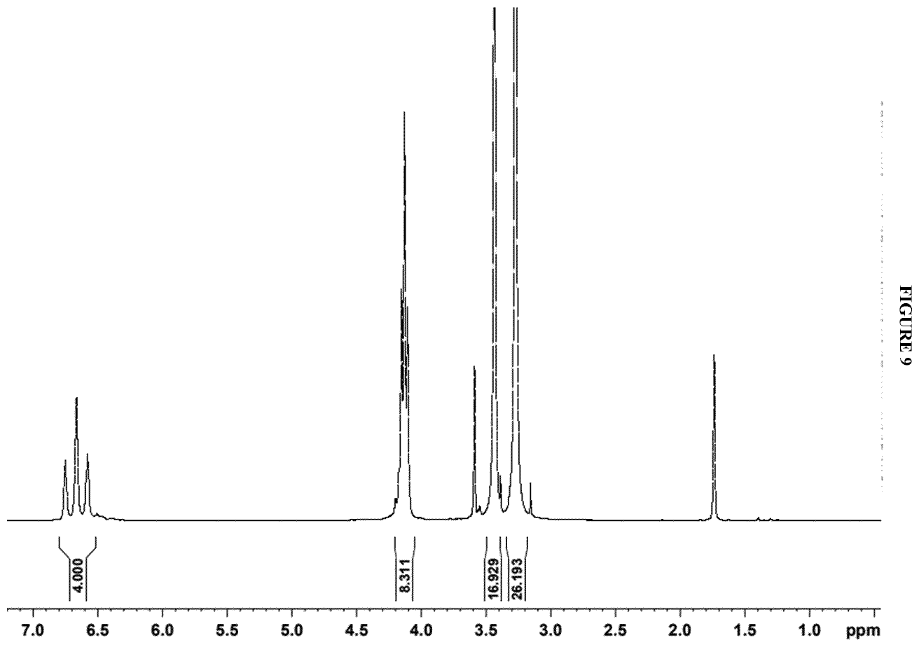

The electrolytes were analysed by ¹H NMR in deuterated THF (FIGS. 5 to 9) and 1,2-DME content was calculated from integrations of the corresponding peaks (3.29 and 3.45 ppm) for four molecules of OFP in the product, which correspond to one lithium cation.

TABLE 1

| | Mass of | | Number of molecule of |
|---|---|---|---|
| | LiAl(OFP)₄ | Volume of 1,2- | 1,2-DME/Li⁺ |
| Electrolyte | weighed (g) | DME added (ml) | determined by NMR |
| 2 | 1.953 | 0.072 | 2.0 |
| 3 | 1.999 | 0.173 | 2.5 |
| 4 | 1.832 | 0.250 | 3.0 |
| 5 | 1.916 | 0.357 | 3.5 |
| 6 | n/a | n/a | 4.25 |

### Compound Example 2

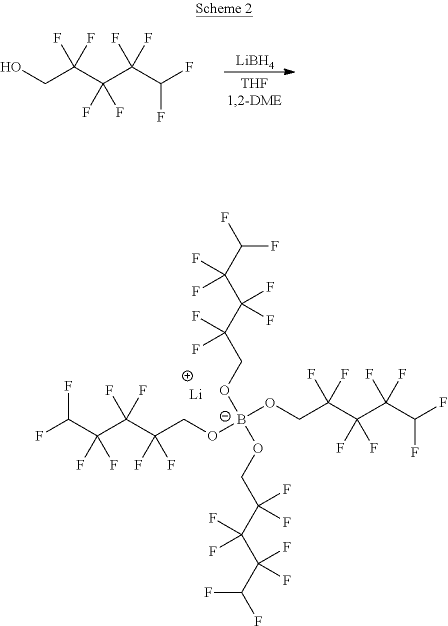 [SMILES: OCC(F)(F)C(F)(F)C(F)(F)C(F)F ; "LiBH4" ; FC(F)C(F)(F)C(F)(F)C(F)(F)CO[B-](OCC(F)(F)C(F)(F)C(F)(F)C(F)F)(OCC(F)(F)C(F)(F)C(F)(F)C(F)F)OCC(F)(F)C(F)(F)C(F)(F)C(F)F ; [Li+]]

The reaction was performed as described in Synthesis 1

To a solution of OFP (5 ml, 35.8 mmol) in anhydrous 1,2-dimethoxyethane (10 ml), a solution of lithium borohydride (4.5 ml, 9.0 mmol, 2.0M in tetrahydrofuran) was added between 5° C. and room temperature. The resulting mixture was stirred at room temperature for 30 minutes, then heated to 60° C. for 1 hour. An in-process check NMR showed unreacted OFP. The solution was cooled down to room temperature and lithium borohydride (0.8 ml, 1.6 mmol, 2.0M in tetrahydrofuran) were added. The solution was heated up to 60° C. for 1 hour and cooled down to room temperature. The excess solvent was removed under reduced pressure (3.2×10⁻² mbar) at 25° C. for 2.5 hours to yield a gel. OFP (0.8 ml, 5.8 mmol) was added and mixture was heated up to 80° C. and 1,2-DME (1.5 ml) were added, the solution was stirred for 4.5 hours, and the solution was then cooled down to room temperature. The excess solvent was then removed under reduced pressure at 80° C. (3.8×10⁻² mbar for 3.5 hours) and the mixture was transferred into an argon glovebox. NMR analysis showed some remaining OFP. Solid was redissolved in 10 ml of 1,2-DME at room temperature, 0.47 ml (0.94 mmol) of lithium borohydride (0.47 ml, 0.94 mmol, 2.0M in tetrahydrofuran) were added drop wise at room temperature. The solution was heated up to 60° C. for 1 hour and lithium borohydride (0.06 ml, 0.12 mmol, 2.0M in tetrahydrofuran) were added dropwise. The mixture was cooled down to room temperature. The excess solvent was then removed under reduced pressure at 25° C. (37.8×10−2 mbar for 30 minutes) to yield a thick oil.

¹H NMR (600 MHz) in deuterated THF: δ (ppm), 3.28 (s, CH₃ from 1,2-DME, 9.71H), 3.44 (s, CH₂ from 1,2-DME, 6.44H), 3.96 (t, CF₂CH₂, J=15.2 Hz, 8H), 6.59 (tt, CF₂CF₂H, J=51.2 Hz, J=5.6 Hz, 4H).

From integration of NMR peaks, it was calculated that for four molecules of OFP in the product, which correspond to one lithium cation there are 1.62 molecules of 1,2-DME present as residual solvent.

### Compound Example 3

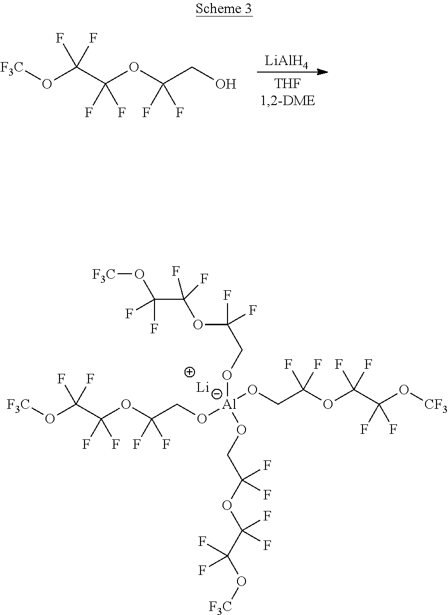

The reaction was performed as described in Synthesis 1

To a solution of fluorinated diethyleneglycol methyl ether (2.75 ml, 17.3 mmol) in anhydrous 1,2-dimethoxyethane (5 ml), a solution of lithium aluminium hydride (4.3 ml, 4.3 mmol, 1.0M in tetrahydrofuran) was added between 15° C. and room temperature. The resulting mixture was stirred at room temperature for 30 minutes, then heated to 60° C. for 30 minutes. The excess solvent was removed under reduced pressure (3.7×10⁻² mbar) at 25° C. for 2 hours to yield a thick gel. Additional 1,2-dimethoxyethane was added to the material to obtain a free-flowing liquid.

¹H NMR (600 MHZ) in deuterated THF: δ (ppm), 3.28 (s, CH₃ from 1,2-DME, 6.24H), 3.44 (s, CH₂ from 1,2-DME, 4.22H), 4.05 (t, CF₂CH₂, J=11.1 Hz, 8H)

From integration of NMR peaks, it was calculated that for four molecules of fluorinated diethyleneglycol methyl ether in the product, which correspond to one lithium cation there are 3.10 molecules of 1,2-DME present as residual solvent.

### Electrochemical Characterisation

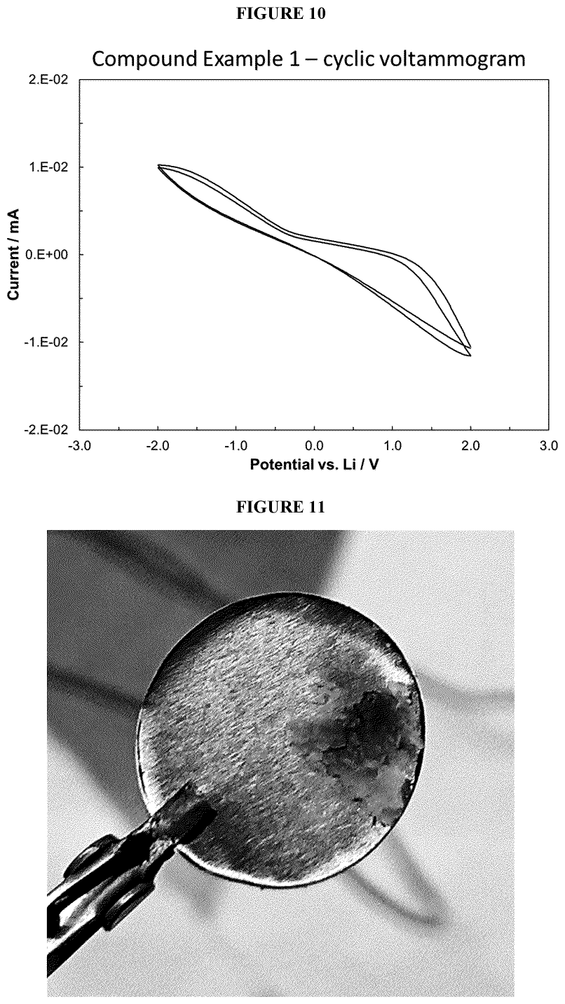

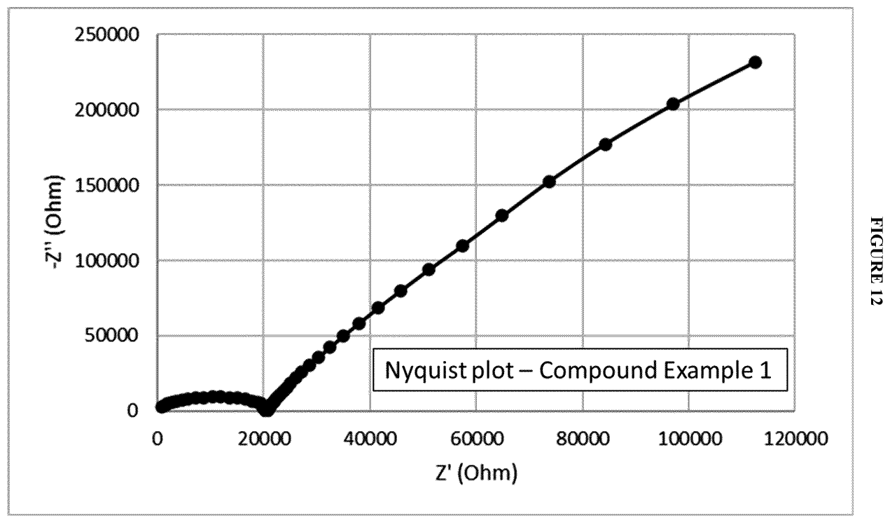

The characterisation of the ionic liquid formed in Synthesis 1 was performed on a 2-electrode cell having a Cu foil (Advent) as the working electrode and a Li foil as the counter electrode/reference electrode, respectively. The ionic liquid was manually deposited between the two electrodes that were connected to a potentiostat (CHI). Cyclic voltammetry measurements were performed to determine the current passing in the cell as a function of applied potential difference at the electrodes. The experiment was performed in an Ar-filled glovebox (MBRAUN). The potential of the Cu electrode was scanned between-2 V and +2 V vs. Li electrode and the cyclic voltammetry plot is shown in FIG. 10. Visual confirmation of Li metal deposition on Cu foil was obtained with a digital picture of the substrate (FIG. 11), in which the darkened area of the image is lithium metal.

### Cell Example 1—Compound of Synthesis 1

EIS measurements were conducted on 2032-type coin cell devices (casings purchased from Cambridge Energy Solutions) having a stainless steel disk (SS), a spacer, made with four layers of Kapton tapes (final thickness 260 microns) which was punched in the middle with a circular hole with a diameter of 0.6 cm. The material containing Compound Example 1, Synthesis 1 was spread to cover the hole. Two more stainless steel disks were placed on top of the stack. The symmetrical cell was assembled in a rigorously dry and oxygen-free Ar-filled MBraun glovebox.

### Cell Example 2—Compound of Synthesis 2

Cells were fabricated by inserting a stainless steel spacer in the bottom of the coin cells described above, followed by a nylon mesh (Merck) having a thickness of 135 microns and a porosity of 47%. 30 microlitres of Electrolyte 1 of Table 1 was drop-cast onto the mesh. The mesh was topped by two stainless steel spacers, a wave spring and the coin cell top, followed by crimping. The cells were assembled in an Argon gas-filled glovebox (MBraun).

### Cell Examples 3-6

Cell Examples 3-6 were formed as described for Cell Example 2 except that Electrolytes 2-5, respectively, of Table 1 were used in place of Electrolyte 1.

### Cell Example 7

The cell was fabricated by inserting a stainless-steel spacer in the coin cell bottom, followed by a fluoro-silicone stencil. The stencil was shaped as a disk of diameter 155 mm, with a circular hole of diameter 5 mm cut in its middle. 30 μl of Compound Example 2 was filled into the hole. On top of the stencil two stainless steel spacers were placed, plus a wave spring and the coin cell top, followed by crimping. The thickness of the stencil in the crimped cell was 360 μm.

### Electrochemical Impedance Spectroscopy (EIS)

EIS measurements were conducted at room temperature. The EIS measurements were taken over a frequency range of 1 Hz to 1 MHz with an amplitude of 5 mV.

Conductivity was calculated using the following formula:

$$σ = \frac{l}{A \cdot R}$$

where 1 is the thickness of the material between the two stainless disks which corresponds to the Kapton spacer thickness of Cell Example 1 (260 microns) and the nylon mesh thickness of Cell Examples 2-6 (135 microns), A is the area of the hole were the material was deposited (diameter 0.6 cm) and R is the impedance. The impedance of the cell was determined by calculating the difference between the second x-axis intercept and the first x-axis intercept in the Nyquist plot, where real impedance (Z′, Ohm) is plotted on the x-axis and the negative imaginary impedance (−Z″, Ohm) is plotted on the y-axis, giving a conductivity of 4.5×10⁻⁶ S/cm.

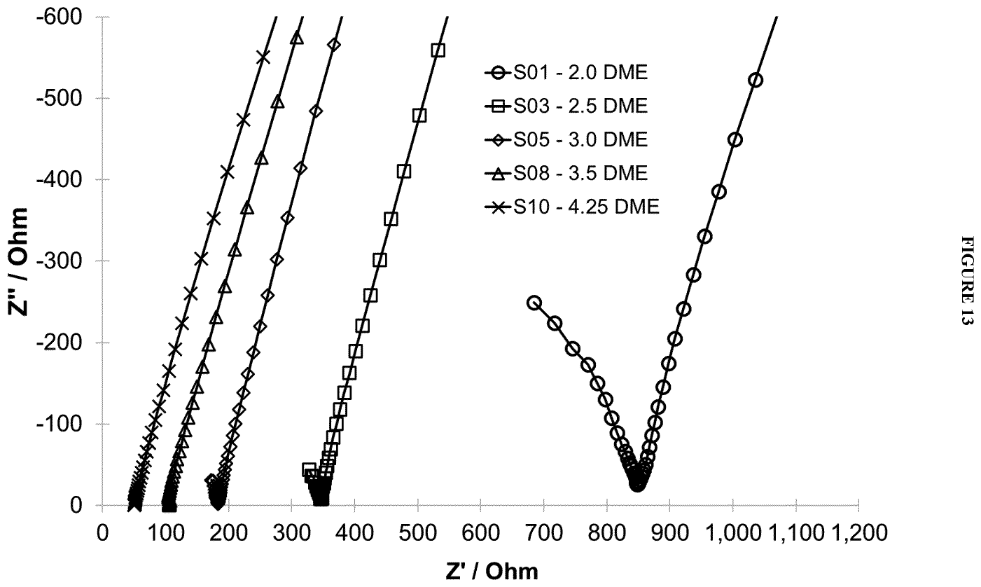

The Nyquist plot for Cell Example 1 is shown in FIG. 12.

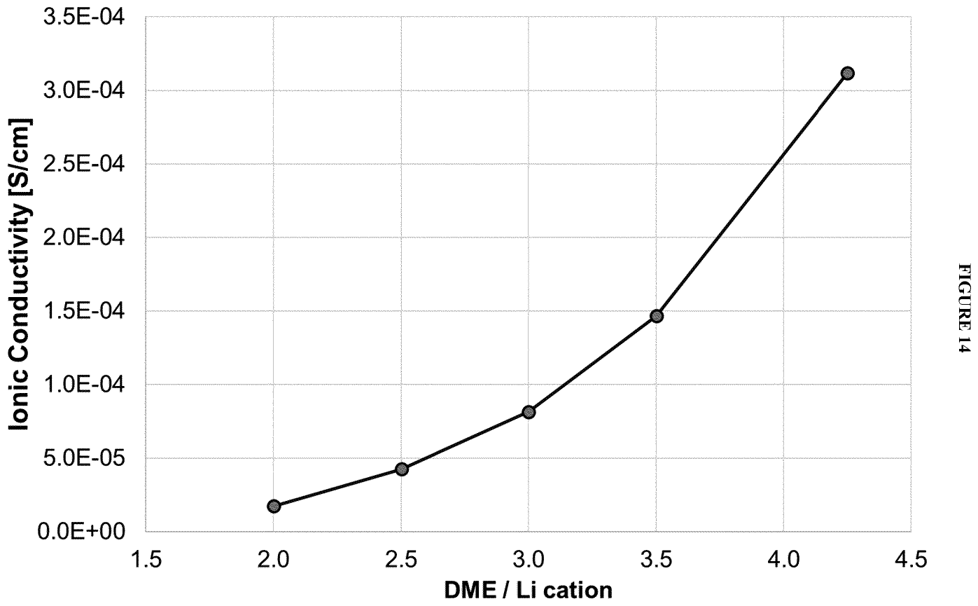

The Nyquist plots for Cell Examples 2-6 are shown in FIG. 13.

Conductivities are given in Table 2

TABLE 2

| | | | | Solvate:Li | |
|---|---|---|---|---|---|
| Cell | | | | cation | Conductivity |
| Example | Compound | Electrolyte | Solvate | ratio | (S/cm) |
| 1 | Compound | 1 | THF | 1 | 4.5 × 10⁻⁶ |
| | Example 1 | | | | |
| | Synthesis 1 | | | | |
| 2 | Compound | 2 | DME | 2 | 1.72 × 10⁻⁵ |
| | Example 1 | | | | |
| | Synthesis 2 | | | | |
| 3 | Compound | 3 | DME | 2.5 | 3.32 × 10⁻⁵ |
| | Example 1 | | | | |
| | Synthesis 2 | | | | |
| 4 | Compound | 4 | DME | 3 | 7.12 × 10⁻⁵ |
| | Example 1 | | | | |
| | Synthesis 2 | | | | |
| 5 | Compound | 5 | DME | 3.5 | 1.04 × 10⁻⁴ |
| | Example 1 | | | | |
| | Synthesis 2 | | | | |
| 6 | Compound | 6 | DME | 4.25 | 2.50 × 10⁻⁴ |
| | Example 1 | | | | |
| | Synthesis 2 | | | | |
| 7 | Compound | n/a | DME | 1.62 | 3.3 × 10⁻⁵ |
| | Example 2 | | | | |

As shown in Table 2 and FIG. 14, increasing the solvate:cation ratio for a given material results in an increase in conductivity.

1. A compound of formula (I): 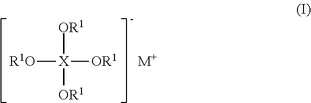
wherein X is Al or B; R¹ in each occurrence is independently a substituent; and two R¹ groups may be linked to form a ring; and M⁺ is a cation.

2. The compound according to claim 1 wherein none of the R¹ groups are linked.

3. The compound according to claim 2 wherein each R¹ is independently a C₁₋₂₀ alkyl group wherein one or more non-adjacent C atoms of the alkyl group may be replaced with O, S, CO or COO and one or more H atoms of the alkyl group may be replaced with F.

4. The compound according to claim 3 wherein each R¹ is independently selected from alkyl and alkyl ether groups wherein one or more H atoms may be replaced with F.

5. The compound according to claim 2 wherein each R¹ is the same.

6. The compound according to claim 1 wherein the compound contains at least 2 different R¹ groups.

7. The compound according to claim 1 wherein R¹ groups of formula (I) are linked and the compound of formula (I) has formula (Ia): 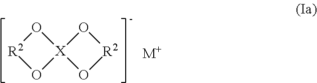
wherein R² in each occurrence is independently a divalent organic group.

8. The compound according to claim 1 wherein M⁺ is an alkali metal ion.

9. The compound according to claim 8 wherein M⁺ is a lithium ion.

10. The compound according to claim 1 wherein M⁺ is a solvated cation.

11. The compound according to claim 10 wherein the solvate of the solvent is selected from solvents comprising at least one ether group.

12. A formulation comprising a compound according to claim 1 and a solvent wherein the solvent:M⁺ molar ratio is no more than 10:1.

13. The formulation according to claim 12 wherein the solvent:M⁺ molar ratio is at least than 0.5:1.

14. A method of forming a compound according to claim 1 comprising reacting a compound of formula (II) and at least one compound selected from formulae (IIIa) and (IIIb):

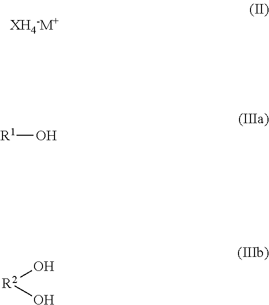

15. The method according to claim 14 wherein the compounds of formula (IIIa) are selected from monohydric alcohols and mono-carboxylic acids and wherein the compounds of formula (IIIb) are selected from diols and di-carboxylic acids.

16. The method according to claim 14 wherein the reaction is carried out in a reaction mixture comprising the solvent of the solvate, wherein M⁺ is a solvated cation.

17. A method comprising at least partially replacing the solvent of the solvate of the compound according to claim 10 with another solvent.

18. A polymer comprising a repeat unit of formula (IV): 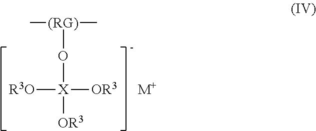
wherein RG is a repeating group of the polymer; R³ is a substituent; X is Al or B; and
M⁺ is a cation.

19. A metal battery or metal ion battery comprising an anode, a cathode and a compound according to claim 1 disposed between the anode and the cathode.

20. (canceled)

21. A metal battery according to claim 19 comprising an anode protection layer comprising the compound or polymer disposed between the anode and cathode.
